(19) United States
(12) Patent Application Publication
(10) Pub. No.: US 2016/0067098 A1
(43) Pub. Date: Mar. 10, 2016

(54) Eye Shield Device

(71) Applicant: Mary A. St. Hilaire, Valrico, FL (US)

(72) Inventor: Mary A. St. Hilaire, Valrico, FL (US)

(21) Appl. No.: 14/482,860

(22) Filed: Sep. 10, 2014

(51) Int. Cl. A61F 9/02

(52) U.S. Cl. CPC A61F 9/029; A61F 9/02; A61F 9/025

(57) ABSTRACT

An eye shield device protects a person's false eyelashes while showering or washing their face. The device includes a shield having a first eye portion coupled to a second eye portion. The first eye portion has a first eye opening positioned therein for aligning with a first eye of a wearer. The second eye portion has a second eye opening positioned therein for aligning with a second eye of the wearer. A pair of eyelash protectors is coupled to the shield and each extends around an associated one of the first and second eye openings. Each of the eyelash protectors extends forwardly away from a first surface of the shield.

### BACKGROUND OF THE DISCLOSURE

### Field of the Disclosure

The disclosure relates to eye covering devices and more particularly pertains to a new eye covering device for protecting a person's eyes, and more particularly, their false eyelashes, while showering or washing the person's face.

### SUMMARY OF THE DISCLOSURE

An embodiment of the disclosure meets the needs presented above by generally comprising a shield having a first eye portion coupled to a second eye portion. The first eye portion has a first eye opening positioned therein for aligning with a first eye of a wearer. The second eye portion has a second eye opening positioned therein for aligning with a second eye of the wearer. A pair of eyelash protectors is coupled to the shield and each extends around an associated one of the first and second eye openings. Each of the eyelash protectors extends forwardly away from a first surface of the shield.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a top front side perspective view of an eye shield device according to an embodiment of the disclosure.

FIG. 2 is a top front side perspective view of an embodiment of the disclosure similar to FIG. 1, except that FIG. 2 shows the visor pivoted upwardly above the eye openings.

FIG. 3 is a front view of an embodiment of the disclosure.

FIG. 4 is a bottom view of an embodiment of the disclosure.

FIG. 5 is a top front side perspective view of an alternative embodiment of the disclosure.

FIG. 6 is a front view of an alternative embodiment of the disclosure.

### DESCRIPTION OF THE PREFERRED EMBODIMENT

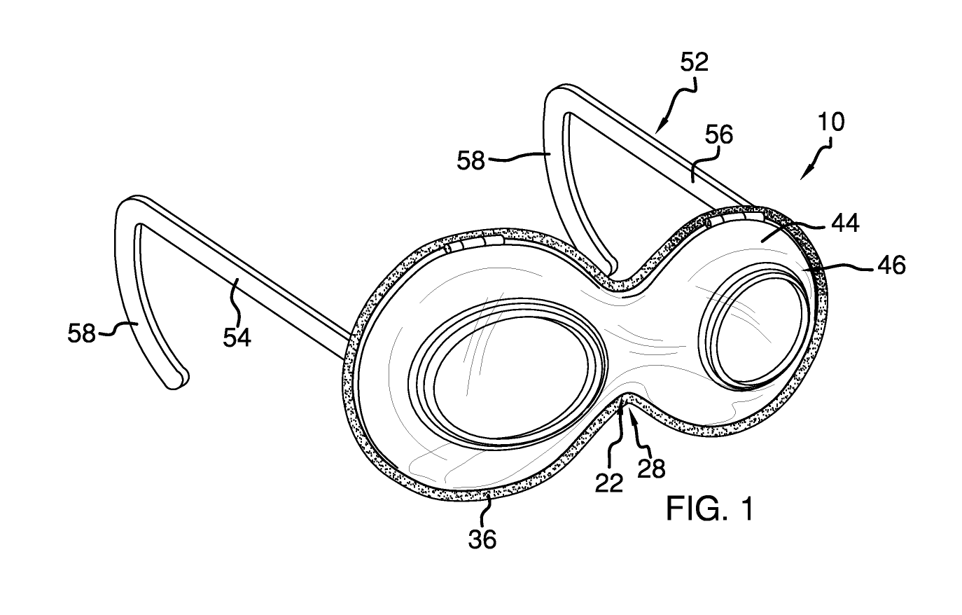

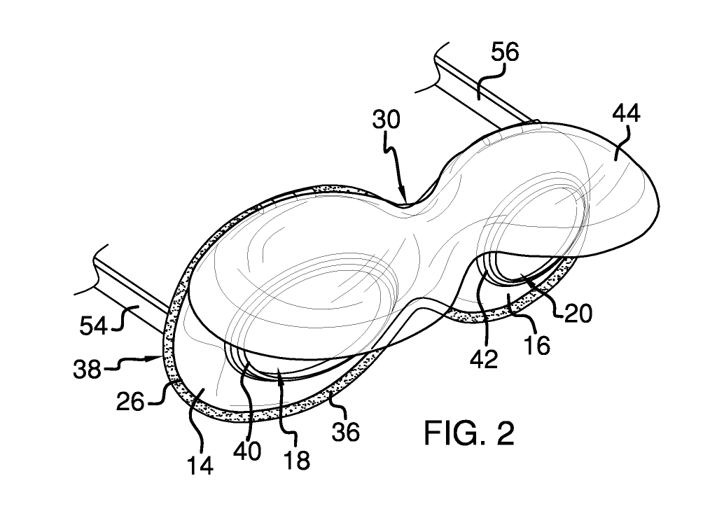

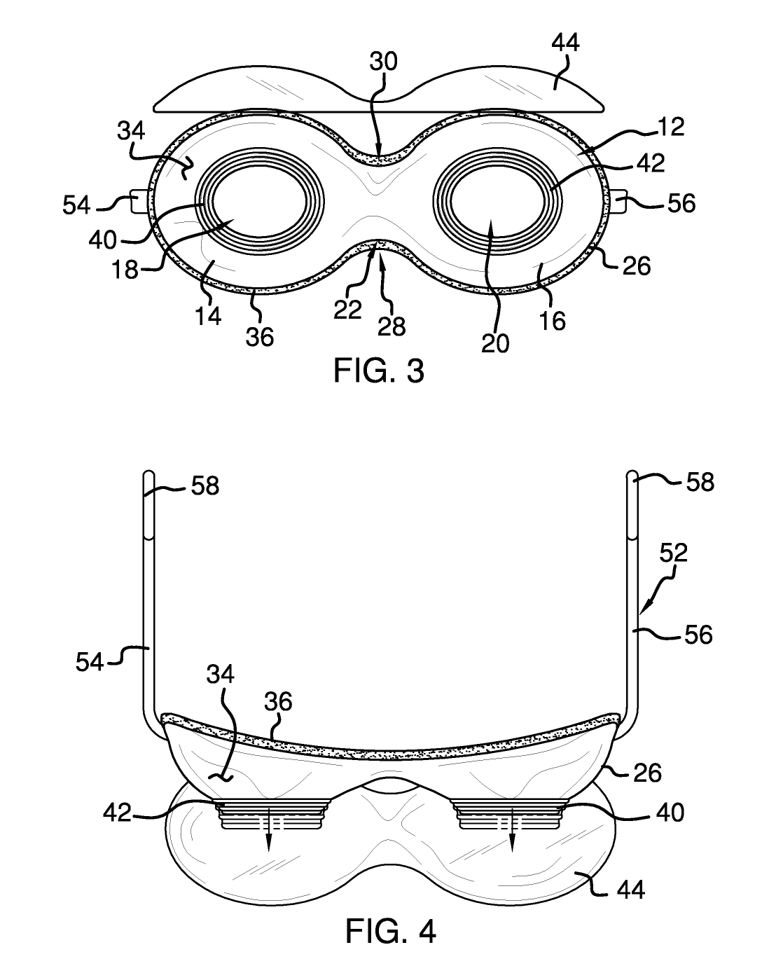

With reference now to the drawings, and in particular to FIGS. 1 through 6 thereof, a new eye covering device embodying the principles and concepts of an embodiment of the disclosure and generally designated by the reference numeral 10 will be described.

As best illustrated in FIGS. 1 through 6, the eye shield device 10 generally comprises a shield 12 having a first eye portion 14 and a second eye portion 16 coupled together. The first eye portion 14 has a first eye opening 18 positioned therein configured for aligning with a first eye of a wearer. The second eye portion 16 has a second eye opening 20 positioned therein configured for aligning with a second eye of the wearer. The shield 12 may be transparent and may contain an anti-fog chemical within an interior space of the shield 12. A first surface 34 of each of the first 14 and second 16 eye portions may be convex extending from a perimeter edge 26 of the shield 12 to each of the first 14 and second 16 eye openings.

The shield 12 has a lower notch 22 extending upwardly into a bottom side 24 of the perimeter edge 26 of the shield 12. The lower notch 22 extends between the first eye portion 14 and the second eye portion 16 defining a nose bridge 28 configured to rest upon a nose of the wearer when the wearer is wearing the shield 12. The shield 12 has an upper notch 30 extending downwardly into a top side 32 of the perimeter edge 26 of the shield 12. The upper notch 30 extends between the first eye portion 14 and the second eye portion 20 and is aligned with the lower notch 22.

A sealant 36 is coupled to and extends around the perimeter edge 26 of the shield 12 wherein the sealant 36 is configured to inhibit a fluid from contacting a second surface 38 of the shield 12 opposite the first surface 34 of the shield 12 when the wearer is wearing the shield 12. The sealant 36 also helps prevent an imprint from being left upon the wearer's face. The sealant 36 may comprise a resiliently compressible material, such as foam or the like.

A pair of eyelash protectors 40, 42 is coupled to the shield 12. Each of the eyelash protectors 40, 42 extends around an associated one of the first 18 and second 20 eye openings and each extends forwardly away from the first surface 34 of the shield 12. Each of the eyelash protectors 40, 42 may be telescopic such that each of the eyelash protectors 40, 42 is forwardly extendable from the shield 12 a selectable distance from the first surface 34 of the shield 12. This allows the eyelash protectors 40, 42 to be adjusted to fit a length of the wearer's false eyelashes so that the wearer's false eyelashes are fully protected from water, shampoo or the like.

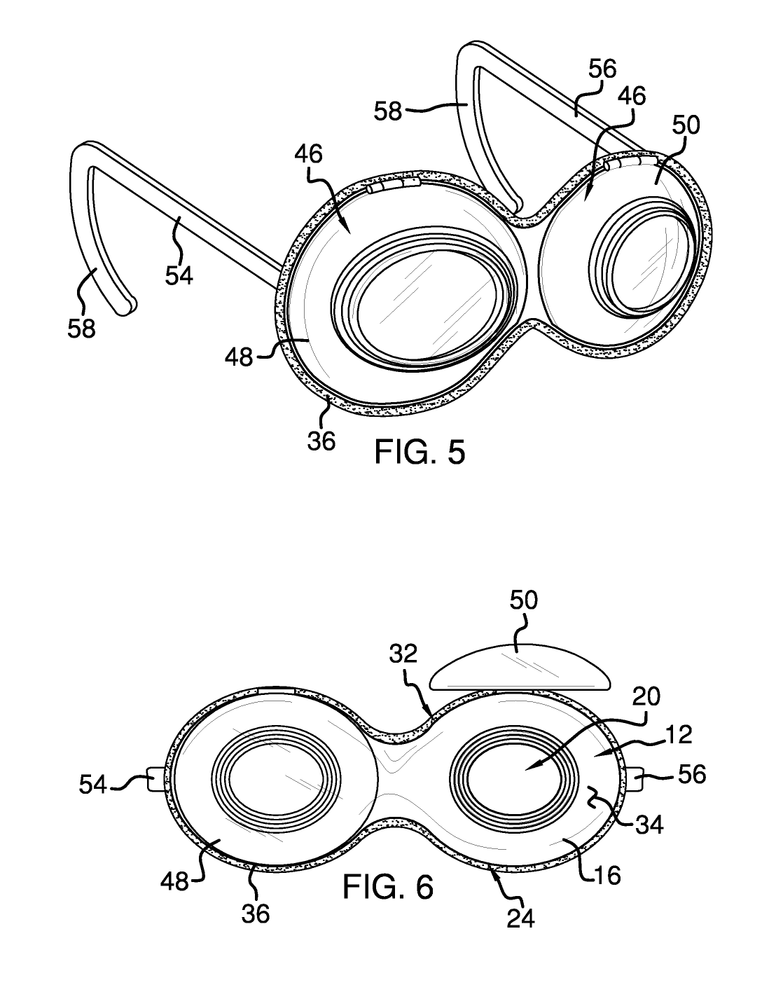

A visor 44 is pivotally coupled to the perimeter edge 26 of the shield 12. The visor 44 may be transparent. The visor 44 is pivotable into a covered position 46 wherein the visor 44 covers each of the first 18 and second 20 eye openings and each of the eyelash protectors 40, 42 when the visor 44 is in the covered position 46. As shown in FIGS. 5 and 6, the visor 44 may include a first visor section 48 and a second visor section 50 pivotally coupled to the shield 12 independently of each other. The first visor section 48 covers the first eye opening 18 when the first visor section 48 is pivoted to the covered position 46. Similarly, the second visor section 50 covers the second eye opening 20 when the second visor section 50 is pivoted to the covered position 46. The first 48 and second 50 visor sections may be spaced from each other.

An ear engagement unit 52 is attached to and extends rearwardly away from the shield 12. The ear engagement unit 52 is configured to engage ears of the wearer. The ear engagement unit 52 may comprise a first temple 54 attached to the first eye portion 14 and a second temple 56 attached to the second eye portion 16. Each of the first 54 and second 56 temples has a distal section 58 relative to the shield 12 that is curved and configured to extend around and engage the ears of the wearer. The first 54 and second 56 temples may be comprised of a firm rubber, plastic or like material.

In use, the shield 12 is positioned over a wearer's eyes so that the first eye opening 18 is positionable in front of a first eye of the wearer and the second eye opening 20 is positionable in front of a second eye of the wearer. The eyelash protectors 40, 42 are extended so that they extend over and fully cover the false eyelashes of the wearer. For additional protection of the wearer's eye region, the visor 44 is pivoted into the covered position 46 to shield against water, shampoo or the like. After use, the device 10 is hung for drying or otherwise stored until needed again.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. An eye shield device comprising:
a shield having a first eye portion and a second eye portion coupled together, said first eye portion having a first eye opening positioned therein configured for aligning with a first eye of a wearer, said second eye portion having a second eye opening positioned therein configured for aligning with a second eye of the wearer; and
a pair of eyelash protectors each being coupled to said shield, each of said eyelash protectors extending around an associated one of said first and second eye openings, each of said eyelash protectors extending forwardly away from a first surface of said shield.

2. The device of claim 1, further comprising an ear engagement unit attached to and extending rearwardly away from said shield, said ear engagement unit being configured to engage ears of the wearer.

3. The device of claim 2, further comprising said ear engagement unit comprising a first temple attached to said first eye portion and a second temple attached to said second eye portion, each of said first and second temples having a distal section relative to said shield being curved and configured to extend around and engage the ears of the wearer.

4. The device of claim 1, further comprising each of said eyelash protectors being telescopic such that each of said eyelash protectors is forwardly extendable from said shield a selectable distance from said first surface of said shield.

5. The device of claim 1, further comprising a visor pivotally coupled to a perimeter edge of said shield, said visor being pivotable into a covered position wherein said visor covers each of said first and second eye openings and each of said eyelash protectors when said visor is in the covered position.

6. The device of claim 1, further comprising a sealant coupled to and extending around a perimeter edge of said shield wherein said sealant is configured to inhibit a fluid from contacting a second surface of said shield opposite said first surface of said shield when the wearer is wearing said shield.

7. The device of claim 6, further comprising wherein said sealant comprises a resiliently compressible material.

8. The device of claim 7, further comprising wherein said resiliently compressible material comprises foam.

9. The device of claim 5, further comprising said visor including a first visor section and a second visor section, each of said first and second visor sections being pivotally coupled to said shield independently of each other, said first visor section covering said first eye opening when said first visor section is pivoted to the covered position, said second visor section covering said second eye opening when said second visor section is pivoted to the covered position.

10. The device of claim 9, further comprising said first and second visor sections being spaced from each other.

11. The device of claim 1, further comprising said shield being transparent.

12. The device of claim 1, further comprising said shield having a lower notch extending upwardly into a bottom side of a perimeter edge of said shield, said lower notch extending between said first eye portion and said second eye portion defining a nose bridge configured to rest upon a nose of the wearer when the wearer is wearing said shield.

13. The device of claim 1, further comprising said shield having an upper notch extending downwardly into a top side of a perimeter edge of said shield, said upper notch extending between said first eye portion and said second eye portion.

14. The device of claim 1, further comprising said first surface of each of said first and second eye portions being convex extending from a perimeter edge of said shield to each of said first and second eye openings.

15. The device of claim 5, further comprising said visor being transparent.

16. An eye shield device comprising:
a shield having a first eye portion and a second eye portion coupled together, said first eye portion having a first eye opening positioned therein configured for aligning with a first eye of a wearer, said second eye portion having a second eye opening positioned therein configured for aligning with a second eye of the wearer, said shield being transparent, said shield having a lower notch extending upwardly into a bottom side of a perimeter edge of said shield, said lower notch extending between said first eye portion and said second eye portion defining a nose bridge configured to rest upon a nose of the wearer when the wearer is wearing said shield, said shield having an upper notch extending downwardly into a top side of said perimeter edge of said shield, said upper notch extending between said first eye portion and said second eye portion, a first surface of each of said first and second eye portions being convex extending from said perimeter edge of said shield to each of said first and second eye openings;
a sealant coupled to and extending around said perimeter edge of said shield wherein said sealant is configured to inhibit a fluid from contacting a second surface of said shield opposite said first surface of said shield when the wearer is wearing said shield, said sealant comprising a resiliently compressible material, said sealant comprising foam;
a pair of eyelash protectors each being coupled to said shield, each of said eyelash protectors extending around an associated one of said first and second eye openings, each of said eyelash protectors extending forwardly away from said first surface of said shield, each of said eyelash protectors being telescopic such that each of said eyelash protectors is forwardly extendable from said shield a selectable distance from said first surface of said shield;
a visor pivotally coupled to said perimeter edge of said shield, said visor being pivotable into a covered position wherein said visor covers each of said first and second eye openings and each of said eyelash protectors when said visor is in the covered position, said visor being transparent; and
an ear engagement unit attached to and extending rearwardly away from said shield, said ear engagement unit being configured to engage ears of the wearer, said ear engagement unit comprising a first temple attached to said first eye portion and a second temple attached to said second eye portion, each of said first and second temples having a distal section relative to said shield being curved and configured to extend around and engage the ears of the wearer.

17. The device of claim 16, further comprising wherein said visor includes a first visor section and a second visor section, each of said first and second visor sections being pivotally coupled to said shield independently of each other, said first visor section covering said first eye opening when said first visor section is pivoted to the covered position, said second visor section covering said second eye opening when said second visor section is pivoted to the covered position, said first and second visor sections being spaced from each other.
